(19) United States
(12) Patent Application Publication
(10) Pub. No.: US 2023/0088478 A1
(43) Pub. Date: Mar. 23, 2023

(54) AUTOMATED METHOD AND SYSTEM FOR INTERPRETING TEST RESULTS

(71) Applicant: Board of Regents, The University of Texas System, Austin, TX (US)

(72) Inventors: Michael Laposata, Galveston, TX (US); Peter McCaffrey, Galveston, TX (US)

(21) Appl. No.: 17/947,899

(22) Filed: Sep. 19, 2022

Related U.S. Application Data
(60) Provisional application No. 63/246,587, filed on Sep. 21, 2021.

(51) Int. Cl. G16H 10/60

(52) U.S. Cl. CPC G16H 10/60

(57) ABSTRACT

A first database comprises test result(s) for patient(s). A second database comprises a database structure having a first data table comprising received interpretation orders, a second data table comprising received test result(s), and a third data table comprising sent interpretation results. An input/output interface receives test result(s) for patient(s) from the first database, and an interpretation order for each test result. Processor(s) automatically store the test result(s) in the second data table, automatically store the interpretation order in the first data table, searches the first data table and the third data table and identifies any interpretation orders that do not have interpretation results sent, automatically obtains health or medical interpretation of the test results in the second data table for each identified interpretation order, automatically sends the health or medical interpretation of the test results to the first database via the input/output interface, and updates the third data table.

### CROSS-REFERENCE TO RELATED APPLICATIONS

This application claims priority to U.S. Provisional Application Ser. No. 63/246,587, filed Sep. 21, 2021 entitled “Automated Method and System for Interpreting Test Results”, which is hereby incorporated by reference in its entirety.

### TECHNICAL FIELD OF THE INVENTION

The present invention relates generally to the field of information systems and, more particularly, to a method and system for automatically interpreting test results.

### INCORPORATION-BY-REFERENCE OF MATERIALS FILED ON COMPACT DISC

Not applicable.

### STATEMENT OF FEDERALLY FUNDED RESEARCH

Not applicable.

### BACKGROUND OF THE INVENTION

Some laboratory information systems have tools that apply generalized text to a class of tests, such as a generalized disclaimer to all HIV test results.

As a result, there is a need for a method and system for automatically interpreting test results.

### SUMMARY OF THE INVENTION

In one embodiment, a computerized method for automatically interpreting one or more test results for one or more patients comprises: providing an input/output interface, a memory, and one or more processors communicably coupled to the input/output interface and the memory; providing a first database and a second database communicably coupled to the one or more processors, wherein the first database contains the one or more test results for the one or more patients, and the second database comprises a database structure having a first data table comprising received interpretation orders, a second data table comprising received test result(s), and a third data table comprising sent interpretation results; receiving at least one of the one or more test results for at least one of the one or more patients from the first database via the input/output interface; automatically storing the received test result(s) for the received patient(s) in the second data table of the second database using the one or more processors; receiving an interpretation order for each received test result via the input/output interface; automatically storing the interpretation order in the first data table of the second database using the one or more processors; searching the first data table and the third data table and identifying any interpretation orders that do not have an interpretation results sent using the one or more processors; automatically obtaining a health or medical interpretation of the received test result(s) for the received patient(s) in the second data table for each identified interpretation order using the one or more processors; and automatically sending the health or medical interpretation of the received test result(s) for the received patient(s) to the first database via the input/output interface, and updating the third data table.

In one aspect, the method receives and stores the at least one of the one or more test results for at least one of the one or more patients in the first database; and automatically generates the interpretation order for the at least one of the one or more test results for at least one of the one or more patients. In another aspect, the interpretation order is only generated when the at least one of the one or more test results is from one or more specified test types. In another aspect, the one or more specified test types comprise a PCR test, an IgG test, or an IgM test. In another aspect, the at least one of the one or more test results comprises a first test result from a first test type and a second test result from a second test type, wherein the first test type and the second test type are different. In another aspect, the first test type or the second test type comprises a PCR test, an IgM test or an IgG test. In another aspect, the at least one of the one or more test results comprises a first test result and a second test result, wherein the first test result is more recent that the second test result. In another aspect, the method receives a verification of the health or medical interpretation prior to automatically sending the health or medical interpretation to the first database. In another aspect, the method receives a change to the health or medical interpretation prior to automatically sending the health or medical interpretation with the change to the first database. In another aspect, the health or medical interpretation comprises text, audio, video or hyperlinks. In another aspect, the health or medical interpretation comprises one or more tests or one or more treatments. In another aspect, the method treats the patient in accordance with the one or more tests or treatments. In another aspect, the method automatically generates a billing claim in accordance with the health or medical interpretation. In another aspect, the method maps one or more parameters of one or more test results to one of the health or medical interpretations. In another aspect, the method automatically exports new test results from the first databases to a shared destination after a specified time period or a specified number of new test results are received.

In another embodiment, a system for automatically interpreting one or more test results for one or more patients comprises: an input/output interface; a memory; one or more processors communicably coupled to the input/output interface and the memory; a first database coupled to the one or more processors, wherein the first database contains the one or more test results for the one or more patients; a second database communicably coupled to the one or more processors, wherein the second database comprises a database structure having a first data table comprising received interpretation orders, a second data table comprising received test result(s), and a third data table comprising sent interpretation results; the input/output interface receives at least one of the one or more test results for at least one of the one or more patients from the first database, and an interpretation order for each received test result; and the one or more processors automatically store the received test result(s) for the received patient(s) in the second data table of the second database, automatically store the interpretation order in the first data table of the second database, searches the first data table and the third data table and identifying any interpretation orders that do not have an interpretation results sent, automatically obtains a health or medical interpretation of the received test result(s) for the received patient(s) in the second data table for each identified interpretation order, automatically sends the health or medical interpretation of the received test result(s) for the received patient(s) to the first database via the input/output interface, and updates the third data table.

In one aspect, the at least one of the one or more test results for at least one of the one or more patients is received and stored in the first database, and the interpretation order for the at least one of the one or more test results for at least one of the one or more patients is automatically generated. In another aspect, the interpretation order is only generated when the at least one of the one or more test results is from one or more specified test types. In another aspect, the one or more specified test types comprise a PCR test, an IgG test, or an IgM test. In another aspect, the at least one of the one or more test results comprises a first test result from a first test type and a second test result from a second test type, wherein the first test type and the second test type are different. In another aspect, the first test type or the second test type comprises a PCR test, an IgM test or an IgG test. In another aspect, the at least one of the one or more test results comprises a first test result and a second test result, wherein the first test result is more recent that the second test result. In another aspect, a verification of the health or medical interpretation is received prior to automatically sending the health or medical interpretation to the first database. In another aspect, a change to the health or medical interpretation is received prior to automatically sending the health or medical interpretation with the change to the first database. In another aspect, the health or medical interpretation comprises text, audio, video or hyperlinks. In another aspect, the health or medical interpretation comprises one or more tests or one or more treatments. In another aspect, the patient is treated in accordance with the one or more tests or treatments. In another aspect, a billing claim in accordance with the health or medical interpretation is automatically generated. In another aspect, one or more parameters of one or more test results are mapped to one of the health or medical interpretations. In another aspect, new test results from the first databases are automatically exported to a shared destination after a specified time period or a specified number of new test results are received.

The present invention is described in detail below with reference to the accompanying drawings.

### BRIEF DESCRIPTION OF THE DRAWINGS

The above and further advantages of the invention may be better understood by referring to the following description in conjunction with the accompanying drawings, in which:

FIG. 1 is a block diagram of a system according to an embodiment of the current invention;

FIG. 2 is a flow chart of a method in according to an embodiment of the current invention;

FIG. 3 is a schematic representation of a non-limiting example of a COVID application and its flow of data according to an embodiment of the current invention;

FIG. 4A is a screen showing a list of test data before a commentary is applied in accordance with an embodiment of the current invention;

FIG. 4B is a screen showing a list of test data after a commentary is applied in accordance with an embodiment of the current invention; and

FIG. 5 is a screen showing an example of a commentary in accordance with an embodiment of the current invention.

### DETAILED DESCRIPTION OF THE INVENTION

The current invention now will be described more fully hereinafter with reference to the accompanying drawings, which illustrate embodiments of the invention. This invention may, however, be embodied in many different forms and should not be construed as limited to the illustrated embodiments set forth herein. For example, the embodiments described herein are not limited to use in a health care environment. Rather, these embodiments are provided so that this disclosure will be thorough and complete, and will fully convey the scope of the invention to those skilled in the art.

Various embodiments of the present invention provide a computerized method and system that applies preformulated clinical interpretive commentary to diagnostic testing. More specifically, this tool allows a user institution to define a collection of test patterns (such as various combinations of COVID PCR and antibody test results) and an accompanying repository of corresponding interpreting content such as explanatory text and to automatically match the appropriate interpretive statement with its corresponding test pattern.

The pre-assignment of clinical interpretive text in test results increases a pathologist's throughput in test interpretation sign out and billable work output. This can accelerate the throughput of an individual pathologist by as much as 10,000% of the national average.

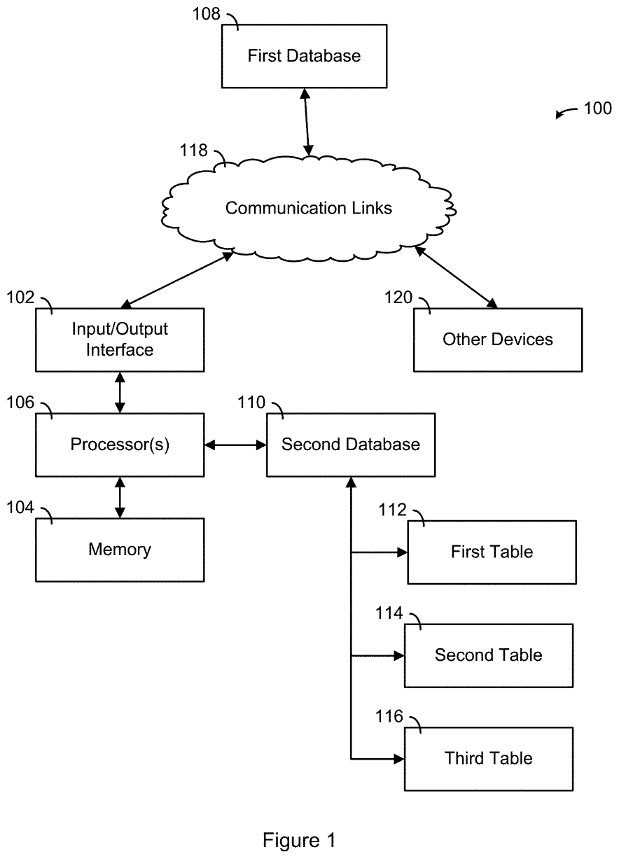

FIG. 1 is a block diagram of a system 100 for automatically interpreting one or more test results for one or more patients in according to an embodiment of the current invention. The system 100 includes an input/output interface 102, a memory 104, one or more processors 106 communicably coupled to the input/output interface 102 and the memory 104, a first database 108 coupled to the one or more processors 106, and a second database 110 communicably coupled to the one or more processors 106. The memory 104 first database 108 and/or second database 110 can be local, remote or distributed. Likewise, the one or more processors 106 can be local, remote or distributed. The first database 108 contains the one or more test results for the one or more patients. The second database 110 comprises a database structure having a first data table 112 comprising received interpretation orders, a second data table 114 comprising received test result(s), and a third data table 116 comprising sent interpretation results. The input/output interface 102 can be any mechanism for facilitating the input and/or output of information (e.g., web-based interface, touchscreen, keyboard, mouse, display, printer, etc.) Moreover, the input/output interface 102 can be a remote device communicably coupled to the one or more processors 106 via one or more communication links 118 (e.g., network(s), cable(s), wireless, satellite, etc.). The one or more communication links 118 can communicably couple the system 100 to other devices 120 (e.g., databases, remote devices, hospitals, doctors, researchers, patients, etc.). The system 100 can be implemented with various devices, such as, server computers, workstation computers, laptop computers, mobile communications devices, personal data assistants, scanning devices or any other devices capable of performing the functions described herein. Note also that the system 100 may include other components not specifically described herein.

The input/output interface 102 receives at least one of the one or more test results for at least one of the one or more patients from the first database 108, and an interpretation order for each received test result. The one or more processors 106 automatically store the received test result(s) for the received patient(s) in the second data table 114 of the second database 110, automatically store the interpretation order in the first data table 112 of the second database 110, searches the first data table 112 and the third data table 116 and identifies any interpretation orders that do not have an interpretation results sent, automatically obtains a health or medical interpretation of the received test result(s) for the received patient(s) in the second data table 114 for each identified interpretation order, automatically sends the health or medical interpretation of the received test result(s) for the received patient(s) to the first database 108 via the input/output interface 102, and updates the third data table 116. Note that “automatically” typically means without human intervention.

In one aspect, the at least one of the one or more test results for at least one of the one or more patients is received and stored in the first database, and the interpretation order for the at least one of the one or more test results for at least one of the one or more patients is automatically generated. In another aspect, the interpretation order is only generated when the at least one of the one or more test results is from one or more specified test types. In another aspect, the one or more specified test types comprise a PCR test, an IgG test, or an IgM test. In another aspect, the at least one of the one or more test results comprises a first test result from a first test type and a second test result from a second test type, wherein the first test type and the second test type are different. In another aspect, the first test type or the second test type comprises a PCR test, an IgM test or an IgG test. In another aspect, the at least one of the one or more test results comprises a first test result and a second test result, wherein the first test result is more recent that the second test result. In another aspect, a verification of the health or medical interpretation is received prior to automatically sending the health or medical interpretation to the first database. In another aspect, a change to the health or medical interpretation is received prior to automatically sending the health or medical interpretation with the change to the first database. In another aspect, the health or medical interpretation comprises text, audio, video or hyperlinks. In another aspect, the health or medical interpretation comprises one or more tests or one or more treatments. In another aspect, the patient is treated in accordance with the one or more tests or treatments. In another aspect, a billing claim in accordance with the health or medical interpretation is automatically generated. In another aspect, one or more parameters of one or more test results are mapped to one of the health or medical interpretations. In another aspect, new test results from the first databases are automatically exported to a shared destination after a specified time period or a specified number of new test results are received.

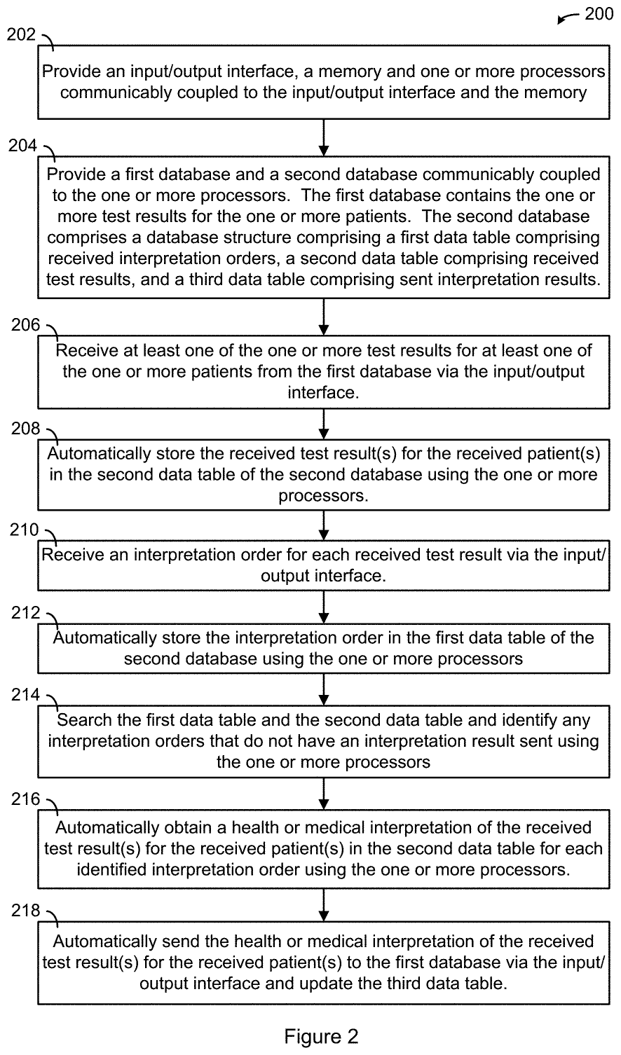

FIG. 2 is a flow chart 200 for managing faculty resources according to an embodiment of the current invention. An input/output interface, a memory, and one or more processors communicably coupled to the input/output interface and the memory are provided in block 202. A first database and a second database communicably coupled to the one or more processors are provided in block 204. The first database contains the one or more test results for the one or more patients. The second database comprises a database structure having a first data table comprising received interpretation orders, a second data table comprising received test result(s), and a third data table comprising sent interpretation results. The at least one of the one or more test results for at least one of the one or more patients is received from the first database via the input/output interface in block 206. The received test result(s) for the received patient(s) are automatically stored in the second data table of the second database using the one or more processors in block 208. An interpretation order for each received test result is received via the input/output interface in block 210. The interpretation order is automatically stored in the first data table of the second database using the one or more processors in block 212. The first data table and the third data table are searched and any interpretation orders that do not have an interpretation result sent are identified using the one or more processors in block 214. A health or medical interpretation of the received test result(s) for the received patient(s) in the second data table is automatically obtained for each identified interpretation order using the one or more processors in block 216. The health or medical interpretation of the received test result(s) for the received patient(s) is automatically sent to the first database via the input/output interface, and the third data table is updated in block 218.

In one aspect, the method receives and stores the at least one of the one or more test results for at least one of the one or more patients in the first database; and automatically generates the interpretation order for the at least one of the one or more test results for at least one of the one or more patients. In another aspect, the interpretation order is only generated when the at least one of the one or more test results is from one or more specified test types. In another aspect, the one or more specified test types comprise a PCR test, an IgG test, or an IgM test. In another aspect, the at least one of the one or more test results comprises a first test result from a first test type and a second test result from a second test type, wherein the first test type and the second test type are different. In another aspect, the first test type or the second test type comprises a PCR test, an IgM test or an IgG test. In another aspect, the at least one of the one or more test results comprises a first test result and a second test result, wherein the first test result is more recent that the second test result. In another aspect, the method receives a verification of the health or medical interpretation prior to automatically sending the health or medical interpretation to the first database. In another aspect, the method receives a change to the health or medical interpretation prior to automatically sending the health or medical interpretation with the change to the first database. In another aspect, the health or medical interpretation comprises text, audio, video or hyperlinks. In another aspect, the health or medical interpretation comprises one or more tests or one or more treatments. In another aspect, the method treats the patient in accordance with the one or more tests or treatments. In another aspect, the method automatically generates a billing claim in accordance with the health or medical interpretation. In another aspect, the method maps one or more parameters of one or more test results to one of the health or medical interpretations. In another aspect, the method automatically exports new test results from the first databases to a shared destination after a specified time period or a specified number of new test results are received.

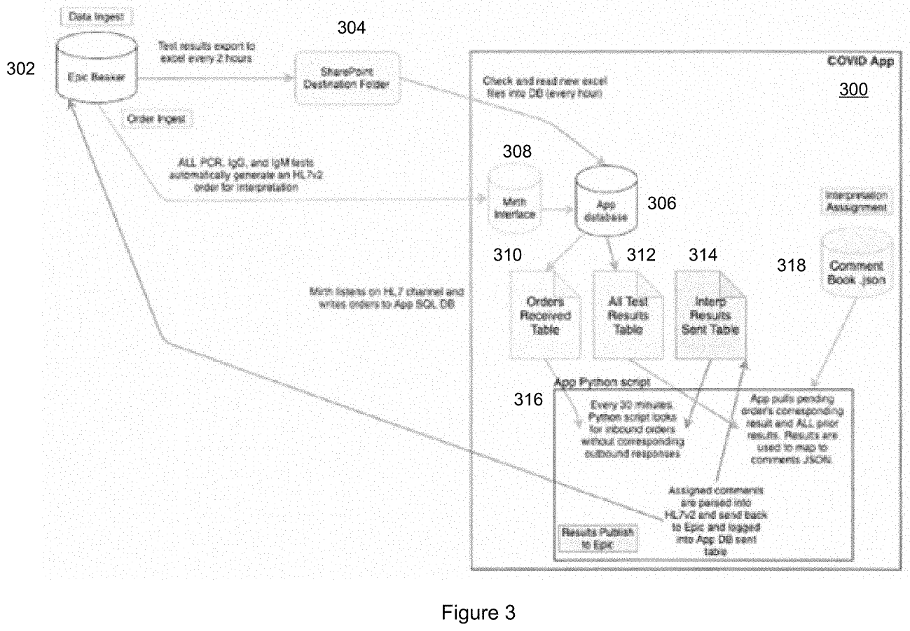

FIG. 3 is a schematic representation of a non-limiting example of a COVID application 300 and its flow of data in accordance with one embodiment of the present invention. Data is ingested into the Epic Beaker 302. Test results are exported to excel every two hours into the SharePoint Destination Folder 304. The COVID application 300 checks and reads new excel files into the App Database 306 every hour. All PCR, IgG and IgM tests automatically generate an HL7v2 order for interpretation that is passed through interface 308 into the App Database 306. The App Database 306 includes an Orders Received Table 310, All Test Results Table 312 and Interp Results Sent Table 314. An App Python script 316 looks for inbound orders without corresponding outbound responses every 30 minutes. The App Python script 316 pulls pending orders corresponding result and ALL prior results. The results are used to map to comments JSON 318. The assigned comments are parsed into HJL7V2, sent back to Epic Beaker 302 and logged into Interp Results Sent Table 314.

The following is a non-limiting example of a comment pertaining to cases where someone has positive COVID testing in the past but is now testing negative where it instructs providers to order repeat testing if symptoms are present:

Interpretation/Recommendations:

Molecular NAAT Tests for Active Infection with the SARS-CoV-2 Virus:

This patient has currently tested negative for the SARS-CoV-2 virus that causes COVID-19 illness. This most likely indicates that the patient does not have an active infection with the SARS-CoV-2 virus. However, infection is not completely ruled out as the false negative rate for molecular NAAT testing using a nasopharyngeal sample can be up to 30%, mostly dependent on the timing of sample collection in relation to illness onset and any deficiencies in sampling techniques. If the patient has symptoms concerning for COVID-19 illness, a repeat NAAT test (PCR, Rapid ID Now, etc.) should be performed, at which time the SARS-CoV-2 virus—if present—may have reached a detectable viral load (usually peaking by the end of the first week of symptoms).

Of note, this patient had a previous infection confirmed by NAAT testing (PCR, Rapid ID Now, etc.), which explains the patient's past positive SARS-CoV-2 IgG test result.

Tests for IgM and/or IgG Antibodies to SARS-CoV-2 Virus:

The patient has previously tested positive for SARS-CoV-2 IgG antibodies, consistent with previous infection. While antibodies to SARS-CoV-2 may provide some degree of immunity, at this time the strength and duration of the antibody response is unknown.

With respect to billing systems, the linkage is automatic but occurs within Epic. When the application is set up, a test is created within Epic for the interpretation itself and it is this test for which the application receives orders and to which the application responds with interpretive comments. Within Epic, this test is linked to generate a G-0459 claim for molecular test interpretation and, from there, it proceeds through Revenue Cycle as with other tests.

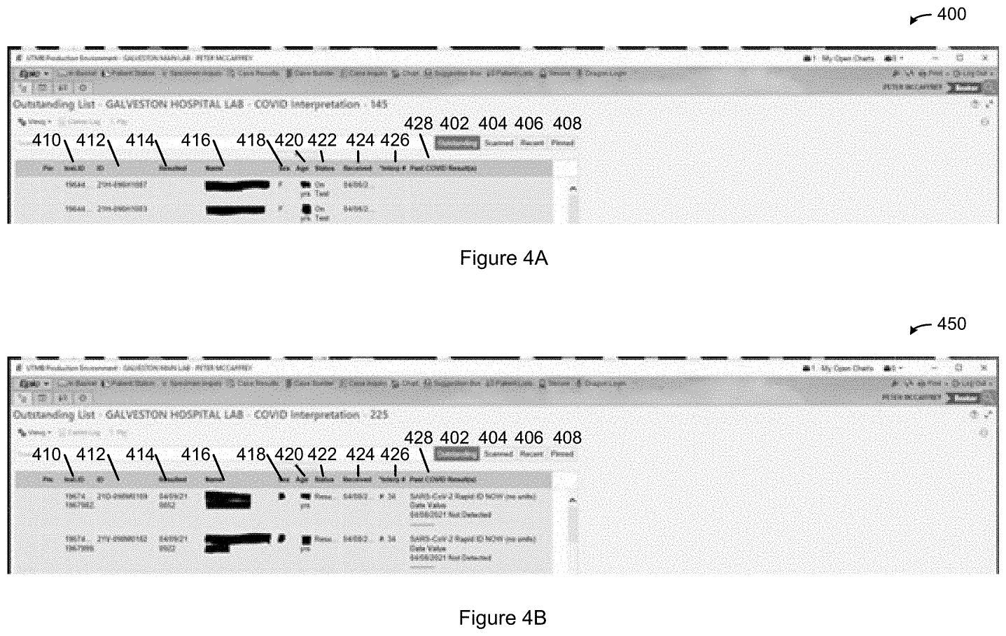

FIG. 4A is a screen showing a list of test data 400 before a commentary (i.e., a health or medical interpretation of the test results) is applied in accordance with an embodiment of the current invention. The pathologist end users see COVID cases coming in to their queue in what is call the “Outstanding List” (tab 402), which is a list of cases that have not yet been signed out. Other tabs include a “Scanned” tab 404, “Recent” tab 406 and “Planned” tab 408. Each test data record includes an institutional identifier 410, a test identifier 412, a date and time that the results were posted 414, a patient name 416, a patient sex 418, a patient age 420, a test status 422, a test order received date 424, an interpretation number 426, and a interpretive comments 428. Notice that the interpretation number column 426 and interpretive comments column 428 are blank. Over time, Pathologists will notice that these cases are automatically annotated with appropriate comment codes. Personal information has been redacted from FIGS. 4A and 4B. Finally, the pathologists verify or alter—where applicable—these outstanding cases to send the results out to patients.

FIG. 4B is a screen showing a list of test data after a commentary (i.e., a health or medical interpretation of the test results) is applied in accordance with an embodiment of the current invention. Interpretive comments 428 end up in the medical record as a specific interpretive result.

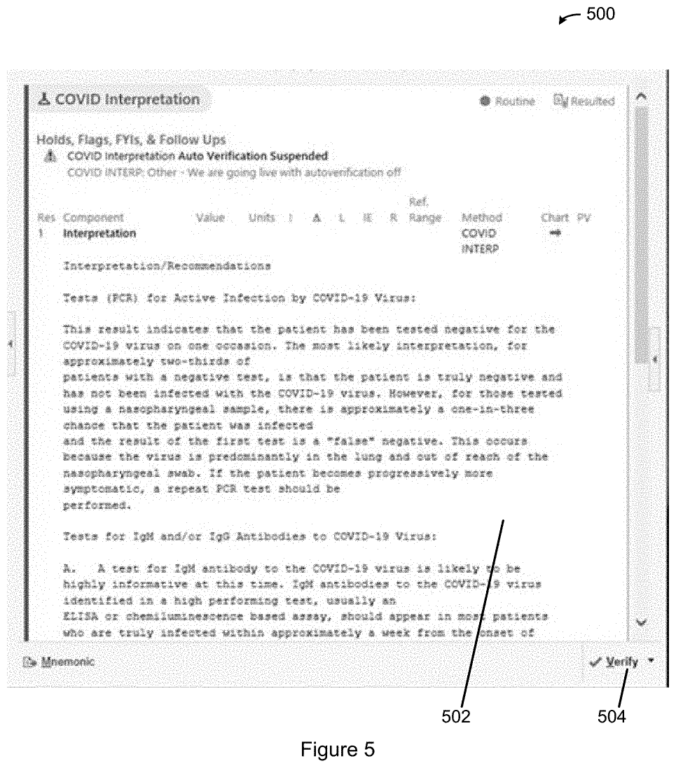

FIG. 5 is a screen 500 showing an example of a commentary (i.e., a health or medical interpretation of the test results) 502 in accordance with an embodiment of the current invention. This figure shows a test result commentary or interpretation that has been automatically generated and is ready for the pathologist to sign-out with the Verify button 504. Note that all of these touchpoints take place within Epic so the UI is already native to those who use the EMR. In this example, the commentary 502 states:
Interpretation/Recommendations
Tests (PCR) for Active Infection by COVID-19 Virus:
This result indicates that the patient has been tested negative for the COVID-19 virus on one occasion. The most likely interpretation, for approximately two-thirds of patients with a negative test, is that the patient is truly negative and has not been infected with the COVID-19 virus. However, for those tested using a nasopharyngeal sample, there is approximately a one-in-three chance that the patient was infected and the result of the first test is a “false” negative. This occurs because the virus is predominantly in the lung and out of reach of the nasopharyngeal swab. If the patient becomes progressively more symptomatic, a repeat PCR test should be performed.
Tests for IgM and/or IgG Antibodies to COVID-19 Virus:
A. A test for IgM antibody to the COVID-19 virus is likely to be highly informative at this time. IgM antibodies to the COVID-19 virus identified in a high performing test, usually an ELISA or chemiluminescence based assay, should appear in most patients who are truly infected within approximately a week from the onset of . . . .

Finally, the application, sits on a server and continuously assigns these interpretations. What you might call an “admin interface” can be shown by logging into the server, seeing what is being assigned. There is an option to update these mappings by updating the associated file on the server that contains them.

To facilitate the understanding of this invention, a number of terms are defined below. Terms defined herein have meanings as commonly understood by a person of ordinary skill in the areas relevant to the present invention. Note that these terms may be used interchangeably without limiting the scope of the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

It will be understood by those of skill in the art that information and signals may be represented using any of a variety of different technologies and techniques (e.g., data, instructions, commands, information, signals, bits, symbols, and chips may be represented by voltages, currents, electromagnetic waves, magnetic fields or particles, optical fields or particles, or any combination thereof). Likewise, the various illustrative logical blocks, modules, circuits, and algorithm steps described herein may be implemented as electronic hardware, computer software, or combinations of both, depending on the application and functionality. Moreover, the various logical blocks, modules, and circuits described herein may be implemented or performed with a general purpose processor (e.g., microprocessor, conventional processor, controller, microcontroller, state machine or combination of computing devices), a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. Similarly, steps of a method or process described herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art.

All of the systems, devices, computer programs, compositions and/or methods disclosed and claimed herein can be made and executed without undue experimentation in light of the present disclosure. While the systems, devices, computer programs, compositions and methods of this invention have been described in terms of various embodiments, it will be apparent to those of skill in the art that variations may be applied to the systems, devices, computer programs, compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A computerized method for automatically interpreting one or more test results for one or more patients comprising:
providing an input/output interface, a memory, and one or more processors communicably coupled to the input/output interface and the memory;
providing a first database and a second database communicably coupled to the one or more processors, wherein the first database contains the one or more test results for the one or more patients, and the second database comprises a database structure having a first data table comprising received interpretation orders, a second data table comprising received test result(s), and a third data table comprising sent interpretation results;
receiving at least one of the one or more test results for at least one of the one or more patients from the first database via the input/output interface;
automatically storing the received test result(s) for the received patient(s) in the second data table of the second database using the one or more processors;
receiving an interpretation order for each received test result via the input/output interface;
automatically storing the interpretation order in the first data table of the second database using the one or more processors;
searching the first data table and the third data table and identifying any interpretation orders that do not have an interpretation results sent using the one or more processors;
automatically obtaining a health or medical interpretation of the received test result(s) for the received patient(s) in the second data table for each identified interpretation order using the one or more processors; and
automatically sending the health or medical interpretation of the received test result(s) for the received patient(s) to the first database via the input/output interface, and updating the third data table.

2. The method of claim 1, further comprising:
receiving and storing the at least one of the one or more test results for at least one of the one or more patients in the first database; and
automatically generating the interpretation order for the at least one of the one or more test results for at least one of the one or more patients.

3. The method of claim 2, wherein the interpretation order is only generated when the at least one of the one or more test results is from one or more specified test types.

4. The method of claim 3, wherein the one or more specified test types comprise a PCR test, an IgG test, or an IgM test.

5. The method of claim 1, wherein the at least one of the one or more test results comprises a first test result from a first test type and a second test result from a second test type, wherein the first test type and the second test type are different.

6. The method of claim 5, wherein the first test type or the second test type comprises a PCR test, an IgM test or an IgG test.

7. The method of claim 1, wherein the at least one of the one or more test results comprises a first test result and a second test result, wherein the first test result is more recent that the second test result.

8. The method of claim 1, further comprising receiving a verification of the health or medical interpretation prior to automatically sending the health or medical interpretation to the first database.

9. The method of claim 1, further comprising receiving a change to the health or medical interpretation prior to automatically sending the health or medical interpretation with the change to the first database.

10. The method of claim 1, wherein the health or medical interpretation comprises text, audio, video or hyperlinks.

11. The method of claim 1, wherein the health or medical interpretation comprises one or more tests or one or more treatments.

12. The method of claim 11, further comprising treating the patient in accordance with the one or more tests or treatments.

13. The method of claim 1, further comprising automatically generating a billing claim in accordance with the health or medical interpretation.

14. The method of claim 1, further comprising mapping one or more parameters of one or more test results to one of the health or medical interpretations.

15. The method of claim 1, further comprising automatically exporting new test results from the first databases to a shared destination after a specified time period or a specified number of new test results are received.

16. A system for automatically interpreting one or more test results for one or more patients comprising:
an input/output interface;
a memory;
one or more processors communicably coupled to the input/output interface and the memory;
a first database coupled to the one or more processors, wherein the first database contains the one or more test results for the one or more patients;
a second database communicably coupled to the one or more processors, wherein the second database comprises a database structure having a first data table comprising received interpretation orders, a second data table comprising received test result(s), and a third data table comprising sent interpretation results;
the input/output interface receives at least one of the one or more test results for at least one of the one or more patients from the first database, and an interpretation order for each received test result; and
the one or more processors automatically store the received test result(s) for the received patient(s) in the second data table of the second database, automatically store the interpretation order in the first data table of the second database, searches the first data table and the third data table and identifying any interpretation orders that do not have an interpretation results sent, automatically obtains a health or medical interpretation of the received test result(s) for the received patient(s) in the second data table for each identified interpretation order, automatically sends the health or medical interpretation of the received test result(s) for the received patient(s) to the first database via the input/output interface, and updates the third data table.

17. The system of claim 16, wherein the at least one of the one or more test results for at least one of the one or more patients is received and stored in the first database, and the interpretation order for the at least one of the one or more test results for at least one of the one or more patients is automatically generated.

18. The system of claim 17, wherein the interpretation order is only generated when the at least one of the one or more test results is from one or more specified test types.

19. The system of claim 18, wherein the one or more specified test types comprise a PCR test, an IgG test, or an IgM test.

20. The system of claim 16, wherein the at least one of the one or more test results comprises a first test result from a first test type and a second test result from a second test type, wherein the first test type and the second test type are different.

21. The system of claim 20, wherein the first test type or the second test type comprises a PCR test, an IgM test or an IgG test.

22. The system of claim 16, wherein the at least one of the one or more test results comprises a first test result and a second test result, wherein the first test result is more recent that the second test result.

23. The system of claim 16, wherein a verification of the health or medical interpretation is received prior to automatically sending the health or medical interpretation to the first database.

24. The system of claim 16, wherein a change to the health or medical interpretation is received prior to automatically sending the health or medical interpretation with the change to the first database.

25. The system of claim 16, wherein the health or medical interpretation comprises text, audio, video or hyperlinks.

26. The system of claim 16, wherein the health or medical interpretation comprises one or more tests or one or more treatments.

27. The system of claim 26, wherein the patient is treated in accordance with the one or more tests or treatments.

28. The system of claim 16, wherein a billing claim in accordance with the health or medical interpretation is automatically generated.

29. The system of claim 16, wherein one or more parameters of one or more test results are mapped to one of the health or medical interpretations.

30. The system of claim 16, wherein new test results from the first databases are automatically exported to a shared destination after a specified time period or a specified number of new test results are received.
